(19) United States
(12) Patent Application Publication
(10) Pub. No.: US 2005/0147670 A1
(43) Pub. Date: Jul. 7, 2005

(54) Oral disintegrating dosage forms

(75) Inventors: Ann F. Hsu, Los Altos Hills, CA (US); Chien-Hsuan Han, Sunnyvale, CA (US)

Correspondence Address: FITCH, EVEN, TABIN & FLANNERY, P. O. BOX 65973, WASHINGTON, DC, 20035, US

(73) Assignee: IMPAX Laboratories Inc., Hayward, CA (US)

(21) Appl. No.: 11/025,013

(22) Filed: Dec. 30, 2004

Related U.S. Application Data
(63) Continuation-in-part of application No. 10/241,837, filed on Sep. 12, 2002.
Application No. 10/241,837 is a continuation-in-part of application No. 10/158,412, filed on May 29, 2002, now abandoned.

(51) Int. Cl. A61K 31/198; A61K 31/137; B27N 3/00

(52) U.S. Cl. 424/464; 514/567; 514/649; 264/109

(57) ABSTRACT

The invention is directed to pharmaceutical dosage forms having immediate release via rapid oral disintegration, specifically, orally disintegrating tablets containing levodopa and carbidopa. The invention further provides formulations containing relatively increased amounts of carbidopa than previously available, including, for example, formulations containing carbidopa-levodopa ratios of about 1:1 to about 1:3.

### CROSS-REFERENCE TO RELATED APPLICATIONS

This application is a continuation-in-part, and claims the benefit under 35 U.S.C. § 120, of U.S. patent application Ser. No. 10/241,837, filed on Sep. 11, 2002, by Chien-Hsuan Han et al., entitled Combination Immediate Release Sustained Release Levodopa/Carbidopa Dosage Forms, which is a continuation-in-part of U.S. patent application Ser. No. 10/158,412, filed on May 29, 2002, by Chien-Hsuan Han et al., entitled Combination Immediate Release Sustained Release Levodopa/Carbidopa Dosage Forms, both of which are incorporated herein by reference in their entirety.

### BACKGROUND OF THE INVENTION

The present invention relates generally to pharmaceutical dosage forms having immediate release via rapid oral disintegration. Specifically, this invention relates to oral disintegrating dosage forms containing levodopa and carbidopa. More specifically, this invention provides for improved formulations that offer increased flexibility and sensitivity in providing patients with optimal levels of levodopa and carbidopa for superior treatment.

The formulations of the present invention are useful for the treatment of several disorders, including Parkinson's disease. Parkinson's disease is a progressive, neurodegenerative disorder of the extrapyrmidal nervous system that affects the mobility and control of the skeletal muscular system. Its medical indications include resting tremor, rigidity, and bradykinetic movements. A symptom of Parkinson's disease is a reduced dopamine level in the patient's brain.

Patients with Parkinson's disease are often treated with levodopa, a metabolic precursor of dopamine, which crosses the blood brain barrier and is rapidly converted into dopamine. Although levodopa rapidly alleviates the symptoms of Parkinson's disease caused by reduced levels of dopamine, treatment may be problematic because it is rapidly decarboxylated by tissues other than the brain. Thus, when levodopa is administered alone, large doses are required because only a small, effective amount of levodopa is transported into the brain.

Aromatic amino acid decarboxylase (AAAD) inhibitors, such as carbidopa, inhibit peripheral levodopa decarboxylation. Hence small doses of carbidopa administered with levodopa allow larger, effective amounts of levodopa to reach the brain and be converted to dopamine. For example, in some studies, carbidopa reduces the amount of levodopa required to produce a given response by about 75 percent and, when administered in conjunction with levodopa, increases plasma levels and the plasma half-life of levodopa.

Pharmaceutical combinations of carbidopa and levodopa are available for the treatment of Parkinson's disease. Such products include SINEMET® tablets and SINEMET® CR sustained release tablets (Bristol-Myers Squibb Co.). Pharmaceutical products containing levodopa and an alternative AAAD inhibitor, such as benserazide, are also available. MADOPARK® (Roche) is one such product. Additionally, some levodopa/carbidopa formulations also include a catechol O-methyltransferase (COMT) inhibitor, such as entacapone, to block the function of another levodopa-degrading enzyme. When entacapone is given in conjunction with levodopa and an AAAD inhibitor, metabolism of levodopa is reduced and plasma levels of levodopa are greater and more sustained compared to administration of levodopa and an AAAD inhibitor alone. For example, when 200 mg of entacapone is administered together with a combination of carbidopa and levodopa, it increases levodopa blood plasma level, measured as the area under the curve (AUC), by about 35 percent, and prolongs the levodopa half-life from 1.3 hours to 2.4 hours. An example of a formulation of carbidopa, levodopa, and entacapone for the treatment of Parkinson's disease is STALEVO® (Novartis Pharmaceuticals USA). Other examples of COMT inhibitors are CGP-28014 and tolcapone.

The dosage forms currently available commercially offer limited dosage flexibility. Typically, current formulations include carbidopa (CD) and levodopa (LD) in ratios of 1:4 or 1:10, for example, in respective amounts 50 mg CD-200 mg LD; 25 mg CD-100 mg LD; 12.5 mg CD-50 mg LD-200 mg entacapone; 25 mg CD-100 mg LD-200 mg entacapone; and 37.5 mg CD-150 mg LD/200 mg entacapone.

Studies suggest that the optimal daily dose for carbidopa is 75 mg to 150 mg per day, on average. One study, however, suggests that additional carbidopa, even in patients receiving “maximally effective” doses of carbidopa, can further enhance the single-dose bioavailability of levodopa. (Cederbaum, J. M., et al., Clinical Neuropharmacology, 1986, 9(2): 153-159). Another study, however, concluded that additional carbidopa produced no additional benefit. (Contin, M., et al., Clinical Neuropharmacology, 1989, 12(1): 75-81).

Low doses of levodopa, in combination with AAAD inhibitors, are used to avoid potential long-term complications associated with high doses of levodopa. With such doses, when the carbidopa-levodopa combination is used in the existing ratios of 1:4 or 1:10, the low doses of levodopa may be associated with insufficient inhibition of peripheral decarboxylase activity, leading to inadequate bioavailability of levodopa and/or unwanted side effects such as nausea or vomiting. Such patients would include levodopa-naïve patients who require dose titration, patients with mild Parkinsonism who may be stabilized with low dose levodopa maintenance regimens, and patients who are treated with low doses of levodopa-carbidopa in combination with other anti-Parkinson's agents such as dopamine agonists or monoamine oxidase inhibitors.

Moreover, despite these available therapies, as Parkinson's disease progresses patients can develop “wearing-off”, a reduction in the duration of their responses and/or an increase in dyskinesias during regular levodopa dosing regimens. Increased dyskinesias may be due to reduced dopamine storage capacity, or less reliable drug absorption as gastrointestinal motility decreases. In these instances, patients may enjoy improved treatment by taking smaller and/or more frequent dosing. Because dosage formulations currently available do not provide sufficient flexibility to adequately tailor dosages to individual patient needs, some patients have been advised to prepare solutions made of carbidopa-levodopa with ascorbic acid as a stabilizer. In many of these cases, the patients may not be taking sufficient carbidopa to provide optimal peripheral decarboxylation.

In addition, it would be desirable to provide an orally disintegrating pharmaceutical dosage formulation of LD and CD which would be convenient for the patient and allow reliable, rapid absorption of the active agents.

In conclusion, there remains a need in the art for dosage formulations that offer various low dosage strengths and different CD-LD ratios, capable of providing levodopa-naïve and late-stage Parkinson Disease patients with increased convenience, easy administration, rapid and reliable absorption, easy dosage adjustment/titration, and better control of dyskinesia or other wearing-off symptoms.

### SUMMARY OF THE INVENTION

The present invention relates generally to pharmaceutical dosage forms comprising carbidopa and levodopa. These dosage forms can be used in the treatment of medical conditions associated with reduced dopamine levels in a patient's brain.

An object of the invention provides for AAAD inhibitor-levodopa dosage forms wherein the AAAD inhibitor-levodopa ratios are from about 1:1 to about 1:3. Preferably the AAAD inhibitor is carbidopa. The carbidopa is present in an amount of at least about 10 mg, preferably at least about 25 mg. These dosage forms are especially useful for levodopa-naïve and late-stage Parkinson Disease patients, other patients maintained at low levodopa doses, or those patients benefiting from dose titration.

A further object of the invention provides an orally disintegrating tablet (ODT) prepared by a wet granulation process comprising: mixing levodopa, carbidopa, a binder, optionally including a further diluent, a disintegrant and/or a sweetener to form a mixture; granulating the mixture with a suitable solvent to form granules; drying the resulting granules; milling oversized granules; blending the resulting granules with a lubricant, and compressing the product into a tablet. Alternatively, the wet granulation may be prepared by mixing the dry ingredients with a solution containing a binding agent.

A further object of the present invention provides for carbidopa-levodopa orally disintegrating tablets, wherein the tablets disintegrate within about 60 seconds in vitro in a USP disintegration apparatus in purified water, and wherein the tablets contain a binder that also functions as a disintegrant. In preferred embodiments, the ODT formulation disintegrates in less than about 45 seconds in vitro in a USP disintegration apparatus in purified water.

In preferred embodiments, the binder is selected from the group consisting of starch, alginic acid, sodium alginate, carboxymethylcellulose sodium, guar gum, hydroxypropyl cellulose, methylcellulose, magnesium aluminum silicate, povidone and gelatin and is present in an amount from about 1 percent by weight to about 80 percent by weight of the total tablet weight. Preferably, the binder is starch and is present in an amount of from about 10 percent to about 70 percent, most preferably, from about 20 percent to about 60 percent by weight of the total tablet weight.

The orally-disintegrating tablets can be prepared by a wet granulation method. In preferred embodiments, the ODT formulation will contain both an intragranular and an intergranular disintegrant. In further embodiments, the ODT formulation will contain a poorly water-soluble filler, such as microcrystalline cellulose, to improve compressibility and rapid dispersion characteristics.

The invention further provides a method of treating patients suffering from Parkinson's Disease or related indications comprising administering an orally-disintegrating tablet comprising carbidopa and levodopa.

### BRIEF DESCRIPTION OF THE FIGURES

FIG. 1 shows the plasma levels of carbidopa and levodopa obtained from 25/250 mg orally disintegrating tablets according to a bioavailability study reported in Example 2.

### DETAILED DESCRIPTION OF THE INVENTION

It should be understood that this invention is not limited to the particular methodology, protocols, and excipients, etc., described herein and as such may vary. The terminology used herein is for the purpose of describing particular embodiments only, and is not intended to limit the scope of the present invention, which is defined solely by the claims.

As used herein and in the claims, the singular forms “a,” “an,” and “the” include the plural reference unless the context clearly indicates otherwise. Thus, for example, the reference to a profile is a reference to one or more such profiles, including equivalents thereof known to those skilled in the art. Other than in the operating examples, or where otherwise indicated, all numbers expressing quantities of ingredients or reaction conditions used herein should be understood as modified in all instances by the term “about.” The term “about” when used in connection with percentages can mean ±1%.

All patents and other publications identified are incorporated herein by reference for the purpose of describing and disclosing, for example, the methodologies described in such publications that might be used in connection with the present invention. These publications are provided solely for their disclosure prior to the filing date of the present application.

Although any known methods, devices, and materials may be used in the practice or testing of the invention, the preferred methods, devices, and materials in this regard are described here.

The present invention relates to new pharmaceutical dosage forms comprising an AAAD inhibitor (such as carbidopa) and levodopa in various dosage strengths. It is designed for the treatment of medical conditions associated with reduced dopamine levels in a patient's brain. Such conditions include symptoms, pathologies, or diseases such as neurological or movement disorders associated with restless leg syndrome, Parkinson's disease, secondary Parkinsonism, Huntingdon's disease, Shy-Drager syndrome, as well as those resulting from brain injury attributable to carbon monoxide or manganese intoxication.

As used herein, the term “patient” means any mammal including humans.

The drugs suitable for use in the pharmaceutical dosage forms according to the present invention include the specified chemical compound as well as salts, analogues, and solvates thereof.

For example, “pharmaceutically acceptable salts” refer to derivatives of the disclosed compounds wherein the specified compound is converted to an acid or base salt thereof. Such pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluensulfonic, methanesulfonic, ethane dislfonic, oxalic, isethionic, and the like.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

The term “derivative” means a chemically modified compound wherein the modification is considered routine by the ordinary skilled chemist, such as an ester or an amide of an acid, protecting groups, such as a benzyl group for an alcohol or thiol, and tert-butoxycarbonyl group for an amine.

The term “effective amount” means an amount of a compound/composition according to the present invention effective in producing the desired therapeutic effect.

Many patients have problems swallowing conventional tablets. These problems lead to poor compliance with treatments and thus have negative impacts on treatment efficiency. See H. Seager, 50 J. Pharm. Pharmacol, 375-382 (1998). Administration of active ingredients through orally-disintegrating tablets solves this problem because quickly disintegrating tablets behave as if the subject were taking a solution or a suspension, i.e., a liquid product. As such, ODT formulations will typically provide rapid and more consistent absorption of the active drug. Thus, it is an object of the present invention to provide a rapidly disintegrating and palatable dosage form over a range of CD-LD doses and dose ratios.

U.S. Pat. No. 6,221,392 (Khankari et al.), the disclosure of which is incorporated by reference, refers to a hard, compressed tablet that dissolves rapidly in the mouth, in which the active ingredient is mixed into a matrix of a non-direct compression filler and a relatively high lubricant content.

U.S. Pat. No. 4,855,326 (Fuisz), the disclosure of which is incorporated by reference, relates to a rapidly dissoluble formulation including a spinnable carrier agent such as a sugar, mixed with a medicament and spun into a fiber.

Levodopa is a known aromatic amino acid precursor of dopamine having the formula (−)-L-α-amino-β-(3,4-dihydroxybenzene) propanoic acid monohydrate. AAAD inhibitors known in the art include carbidopa, benserazide, alpha-monofluoromethyldopa, and 3-hydroxybenzylhydrazine.

The active ingredients of the present inventions, levodopa and, preferably, carbidopa, are present in a ratio of from about 1:1 to about 1:10. Importantly, the present invention provides dosage forms with higher ratios of AAAD inhibitor to levodopa than are currently available. In particular, ratios of AAAD inhibitor, e.g., carbidopa, to levodopa in the range of 1:1 to 1:3 are provided. In contrast, commercially available products have proportionally less AAAD inhibitors, in that they contain ratios of 1:4 or 1:10 AAAD inhibitor to levodopa.

Studies suggest that a daily dose for carbidopa of 75 mg to 150 mg per day is required to maximally inhibit systemic dopa decarboxylase. One study, however, suggests that additional carbidopa, even in patients receiving 125 mg to 175 mg doses of carbidopa, can further enhance the bioavailability of levodopa. (Cederbaum, J. M., et al., Clinical Neuropharmacology, 1986, 9(2): 153-159). Another study, however, concluded that additional carbidopa produced no additional benefit. (Contin, M., et al., Clinical Neuropharmacology, 1989, 12(1): 75-81). Taken together, the results of these studies support that optimum doses of carbidopa (or other AAAD inhibitors) vary significantly among individuals who are treated with levodopa.

Low doses of levodopa, in combination with AAAD inhibitors, are increasingly being used to avoid potential long-term complications associated with high doses of levodopa. When the carbidopa-levodopa combination is used in the existing ratios of 1:4 or 1:10, low doses of levodopa may be associated with insufficient inhibition of peripheral decarboxylase activity, leading to inadequate bioavailability of levodopa and/or unwanted side effects such as nausea or vomiting. Patients taking low doses of levodopa would include levodopa-naïve patients who require dose titration, patients with mild Parkinsonism who may be stabilized with low and less frequent maintenance levodopa regimens, and patients who are treated with low doses of levodopa-carbidopa in combination with other anti-Parkinson's agents such as dopamine agonists or monoamine oxidase inhibitors.

Therefore, the formulations of the present invention containing AAAD inhibitor to levodopa in ratios ranging from 1:1 to 1:3 will provide proportionally more AAAD inhibitor, such that patients taking low doses of levodopa will obtain sufficient daily AAAD inhibitor to adequately inhibit peripheral decarboxylase activity and improve the bioavailability of levodopa. Preferably, the AAAD inhibitor is carbidopa. Ideally, a range of dosages are provided containing fixed-dosage amounts of carbidopa. These dosage forms contain at least about 15 mg, preferably about 25 mg carbidopa. In this aspect of the invention, preferred dosages contain 25 mg carbidopa-25 mg levodopa; 25 mg carbidopa-50 mg levodopa; or 25 mg carbidopa-75 mg levodopa.

The pharmaceutical dosage form may be a particle, a tablet, or a layered tablet. Dosage forms can be made according to known methods in the art.

Orally-Disintegrating Tablets

Preparation of quickly-disintegrating oral dosage forms containing carbidopa and levodopa presents certain difficulties associated with the physical properties of carbidopa and levodopa, such as their low water solubility. In addition, their physical properties make it difficult to prepare a direct blend formulation with good flow properties.

As a result, the present inventors have developed an ODT formulation which is easy to manufacture using standard wet granulation processes known to those of skill in the art. The resulting formulation has good hardness, yet retains the rapidly-disintegrating quality that is desirable in an ODT formulation. ODT formulations prepared in accordance with many prior art methods have high friability, meaning that they are often required to be shipped in blister packs to minimize breakage. In contrast, the ODT formulations of the present invention will typically have good hardness, which will allow use of traditional high-speed tablet presses and storage and shipment in bulk containers or bottles.

Typically, the ODT formulations of the present invention will have a hardness of 2 to 7 kp, usually greater than 3 kp.

The orally-disintegrating tablets of the present invention will disintegrate in the mouth or, in vitro in a USP disintegration apparatus in purified water within 60 seconds, preferably within about 45 seconds. Disintegration time is determined by placing individual tablets within a tube in a USP basket-rack assembly, which basket is raised and lowered into purified water maintained at 37° C., at a constant frequency rate of between 29 and 32 cycles per minute. The disintegration rate is the time in which the tablets are disintegrated completely.

Binders are one or more ingredients that are added to form granules and/or promote cohesive compacts during compression. The ODT formulation of the present invention incorporates a binder that is strong enough to enable the formation of a tablet, but not so strong as to retard disintegration. Preferably the binder is one that also has disintegrant properties. As such, preferred binders include starch, alginic acid, sodium alginate, carboxymethylcellulose sodium, guar gum, hydroxypropyl cellulose, methylcellulose, magnesium aluminum silicate, povidone, gelatin and a mixture of any thereof. Preferably the binder is starch.

The binder will typically be present in about one percent to about 80 percent by weight of the total tablet weight. When the binder is starch, it will typically be present in about 10 percent to about 70 percent by weight of the total tablet weight. Preferably, the binder will be present in about 20 percent to about 60 percent by weight of the total tablet weight. When the binder comprises starch, any pharmaceutically acceptable starch may be used in the present invention, including potato starch, rice starch, corn starch and pregelatinized starch, in molecular weights as described in the “Handbook of Pharmaceutical Excipients,” Eds. A. Wade and P. J. Weller (2nd ed. 1994), Joint publication of American Pharmaceutical Association, Washington, USA and The Pharmaceutical Press, London, England, the disclosure of which regarding binders, including starch, is incorporated herein by reference.

Diluents that are useful in the present invention may include any conventional diluents known in the art, provided they have good compressibility. At least some of the diluent used in the ODT formulation should be poorly water-soluble, which will improve dispersion. This is due, in part, to the low water solubility of carbidopa and levodopa. By contrast, highly soluble fillers such as saccharides (e.g., lactose and mannitol) should be kept to a minimum, as their use will lead to the formation of large granules in a wet granulation process, which can contribute to an unpleasant “gritty” mouth feel and slower dissolution of an ODT formulation.

Compressible, poorly water-soluble, diluents useful in the present invention include cellulose and cellulose derivatives such as methylcellulose, carboxymethylcellulose, microcrystalline cellulose and the like. The diluent may be present in an amount of up to about 70 percent by weight of the total tablet weight. Preferably, the diluent will be present in an amount up to about 60 percent by weight of the total tablet weight, usually less than 50 percent by weight of the total tablet weight, depending on the amount of binder used. A preferred diluent is microcrystalline cellulose.

In order to obtain the desired rapidly disintegrating quality of the ODT formulation, disintegrants will typically be included in the formulation. Some disintegration functionality may be provided by the binder, for example, where the binder is starch. Additional disintegrants will also be beneficial in the ODT formulation of the present invention. In a wet granulation process, a disintegrant may be included in the preparation of the granules, i.e., an intragranular disintegrant, or a disintegrant may be added to the prepared granules, prior to pressing into tablets, i.e., an intergranular disintegrant. The intergranular disintegrant will assist in the disintegration of the tablet back into granules, whereas the intragranular disintegrant will assist in the disintegration of the granules themselves. Preferably, the formulation contains both an intragranular and an intergranular disintegrant.

Disintegrants that are useful in the orally disintegrating tables of the present invention include, for example, croscarmellose sodium, kaolin, powdered sugar, crospovidone (cross-linked PVP), carboxymethylcellulose, alginic acid, sodium alginate, polacrilin potassium, sodium starch glycolate, hydroxypropyl cellulose, methylcellulose, magnesium aluminum silicate microcrystalline cellulose and the like, including a mixture of any thereof. The disintegrant may be present in an amount of about 0.1 percent to about 10 percent by weight of the total tablet weight. Preferably, an intragranular disintegrant will be present in about one percent to about five percent by weight of the total tablet weight, and an intergranular disintegrant will be present up to about five percent by weight of the total tablet weight. A preferred intragranular disintegrant is croscarmellose sodium and a preferred intergranular disintegrant is crospovidone.

The orally disintegrating tablet formulation may also include a lubricant, such as, for example, a lubricant selected from the group consisting of talc, magnesium stearate, calcium stearate, zinc stearate, stearic acid, sodium stearyl fumarate, micronised polyoxyethyleneglycol, leukine, sodium benzoate, alkaline stearates, mineral and vegetable oils, glyceryl behenate and sodium stearyl fumarate and a mixture of any thereof. Further, the pharmaceutical dosage form will typically also include a sweetener, such as, for example, a sweetener selected from the group consisting of aspartame, potassium acesulfame, sodium saccharinate, neohesperidin dihydrochalcone, sucralose, monoammonium glycyrrhizinate, and a mixture of any thereof.

The pharmaceutical dosage form also may contain any other excipients with dissolution features consistent with the objectives herein. Other water soluble excipients may include saccharides, such as lactose or mannitol, in limited amounts.

The orally disintegrating tablet formulation may contain additional pharmaceutically acceptable carriers, diluents, excipients, or vehicles, such as preserving agents, polymers, glidants, wetting agents, emulsifying agents, suspending agents, sweetening agents, flavoring agents, coloring agents, perfuming agents, lubricating agents, acidifying agents, and dispensing agents. Such ingredients, including pharmaceutically acceptable carriers and excipients that may be used to formulate oral dosage forms, are described in the Handbook of Pharmaceutical Excipients, American Pharmaceutical Association (1986), incorporated herein by reference in its entirety.

A wicking agent may also be used. Wicking agents are compositions which are capable of drawing water up into the dosage form. They help transport moisture into the interior of the dosage form. In that way the dosage form can dissolve from the inside, as well as from the outside. Any chemical which can function to transport moisture can be considered a wicking agent. Wicking agents include microcrystalline cellulose (AVICEL PH 200, AVICEL PH 101), Ac-Di-Sol (croscarmellose sodium) and PVP-XL (a crosslinked polyvinylpyrrolidone); starches and modified starches, polymers, and gum such as arabic and xanthan. Hydroxyalkyl cellulose such as hydroxymethylcellulose, hydroxypropylcellulose and hydroxyopropylmethylcellulose, as well as compounds such as carbopol may be used as well.

Determination of workable proportions in any particular instance will generally be within the capability of the person skilled in the art. Details concerning any of the excipients of the invention may be found in Fiedler, H. P. “Lexikon der Hilfsstoffe fur Pharmazie, Kosmetik und angrenzende Gebiete,” Editio Cantor Verlag Aulendorf, Aulendorf, (4th ed. 1996); “Handbook of Pharmaceutical Excipients,” Eds. A. Wade and P. J. Weller (2nd ed. 1994), Joint publication of American Pharmaceutical Association, Washington, USA and The Pharmaceutical Press, London, England; the contents of which are hereby incorporated by reference. Details concerning a commercially available excipient may be obtained from the relevant manufacturers. Detailed methods for creating orally-disintegrating or dispersable tablets are described in U.S. Pat. Nos. 6,733,781, 6,723,348, 6,024,981 and 6,376,545, each of which is hereby incorporated entirely by reference.

Disintegration time in the mouth can be measured by observing the dissolution time of the tablet in, for example, purified water at about 37° C., using the USP basket-rack assembly method.

The dosage form of the present invention disintegrates within 60 seconds and provides an immediate release of levodopa and carbidopa, to provide early relief from symptoms via quick onset of effective blood plasma levels of active agent. Preferably, the orally-disintegrating tablet disintegrates within about 45 seconds.

A COMT inhibitor may be included with the AAAD inhibitor and levodopa in the dosage forms of the present invention. COMT inhibitors include CGP-28014, entacapone, and tolcapone. The use of entacapone has been studied in conjunction with carbidopa/levodopa therapy in patients with Parkinson's disease. See Ahtila et al., “Effect of Entacapone, A COMT Inhibitor, on the Pharmacokinetics and Metabolism of Levodopa After Administration of Controlled release Levodopa-Carbidopa in Volunteers,” Clinical Neuropharmacology, 18(1), 46-57 (1995). U.S. Pat. No. 6,500,867 (Virkki et al.) discloses formulations containing levodopa, carbidopa, and entacapone. Other examples of COMT inhibitors are CGP-28014 and tolcapone. U.S. Pat. No. 6,797,732 discloses COMT inhibitors in combination with levodopa/carbidopa. The disclosures of the foregoing journal article and patents are incorporated herein by reference.

Yet another embodiment of the present invention may be a pharmaceutical dosage form that further comprises one or more drugs selected from the group consisting of anti-cholinergics, beta 2-agonists, cyclooxygenase-2 (COX-2) inhibitors, dopamine receptor agonists, monoamine oxidase (MAO) inhibitors, opiate delta receptor agonists, opiate delta receptor antagonists, and N-methyl-D-aspartate (NMDA) antagonists. One other embodiment may also include one or more drugs selected from the group consisting of albuterol, alpha-lipoic acid, amantadine, andropinirole, apomorphine, baclofen, biperiden, benztropine, bromocriptine, budipine, cabergoline, clozapine, deprenyl, dextromethorphan, dihydroergokryptine, dihydrolipoic acid, eliprodil, eptastigmine, ergoline, formoterol, galanthamine, lazabemide, lysuride, mazindol, memantine, methylphenidate, mofegiline, orphenadrine, pergolide, pirbuterol, pramipexole, propentofylline, procyclidine, rasagiline, remacemide, riluzole, rimantadine, ropinirole, salmeterol, selegiline, spheramine, terguride, and trihexyphenidyl.

The dosage forms of the present invention include a wide variety of possible combinations of amounts of levodopa and carbidopa. Total daily dosages of the compounds useful according to this invention administered to a patient are generally in amounts of from about 0.01 mg/kg to about 100 mg/kg body weight daily, for example from about 0.05 mg/kg to about 50 mg/kg body weight daily. Both the levodopa and carbidopa doses fall within this mg/kg/day dosage range. The skilled artisan will appreciate that daily dosages having an amount of active agent sufficient to treat Parkinson's disease will generally contain from about 25 mg to about 4,000 mg levodopa in combination with from about 5 mg to about 600 mg carbidopa. Preferably, the total daily dosage of carbidopa will be at least about 75 mg.

Such dosage forms may contain from about 25 to about 600 mg levodopa in combination with from about 10 to about 200 mg carbidopa. Other dosage forms contain 25, 37.5, 50, 70, 75, 80, 100, 125, 130, 150, 200, 250, 300, 400, or 600 mg levodopa and 12.5, 25, 37.5, 50, 62.5, 75, 100, 112.5, 125 or 150 mg carbidopa. Preferred CD-LD dosage forms include, respectively, 25-25, 25-50, 25-75, 25-100, 25-125, 25-150, 25-200, 25-250 and 10-100 mg per tablet of carbidopa and levodopa.

Dosage unit compositions may also contain amounts of levodopa and carbidopa in percentages of these dosages as may be used to make up the daily dose. It should be understood, however, that the specific dose level for any particular patient will depend upon a variety of factors including body weight, general health, gender, diet, time of administration, rates of absorption and excretion, combination with other drugs, and the severity of the particular disease being treated. Actual dosage levels of active ingredient in the compositions of the present invention may be varied so as to obtain an amount of active ingredient that will obtain an effective dosaging regimen for the desired therapeutic response.

The orally disintegrating tablets of the present invention may be manufactured using wet granulation processes known in the art. In general, the active ingredients are mixed thoroughly with the binder, filler, a disintegrant and, optionally, a sweetener. Additional taste-masking substances may also be included. The mixture is granulated with purified water, or other suitable solvent, in a granulator such as a high shear granulator (e.g., a PMA-65), a fluidized bed granulator, a planetary mixer, or an extruder, to form granules. Alternatively, a solution of the binder, such as starch or gelatin in water, may be added to a mixture of dry ingredients prior to granulation. The wet granules are then dried, usually until the Loss on Drying (LOD) is less than or equal to 3%. The dried granules are then screened and milled to a uniform size. The screened and milled granules may be blended with a lubricant, with or without additional disintegrant. The mixture may be pressed into tablets under conventional conditions, for example, using a high-speed tablet press, such as a Manesty betapress. The compressed tablets may be film coated using standard ingredients and procedures known to those of skill in the art of pharmaceutical science.

An aspect of the invention includes a method of treating a patient in need of treatment for Parkinson's Disease or related indications. An exemplary such method comprises administering to such a patient an oral disintegrating dosage (ODT) form of our invention.

Without further elaboration, one skilled in the art having the benefit of the preceding description can utilize the present invention to the fullest extent. The following examples are illustrative only and do not limit the scope of our inventions in any way.

### EXAMPLES

### Example 1

### Orally Disintegrating Dosage Form

| Ingredient | Percent by weight |
|---|---|
| Carbidopa, USP | 6.75 |
| Levodopa, USP | 25.05 |
| Aspartame | 5.00 |
| Microcrystalline cellulose, NF | 28.50 |
| Corn starch, NF (Pure-Dent B700, | 28.50 |
| Grain Processing Corp) | |
| Croscarmellose sodium, NF | 2.85 |
| Crospovodone, NF | 1.90 |
| Magnesium stearate | 1.44 |
| Purified water, USP | NA |

All ingredients, except magnesium stearate and crospovidone, were weighed and mixed thoroughly. The mixed ingredients were granulated in a high shear granulator with purified water, and the granules were dried overnight in an oven at 60° C. The dry granules were screened through a 25 US mesh screen, and the oversized granules were milled through a #20 US mesh screen. The screened and milled granules were blended with crospovidone, followed by addition of magnesium stearate, and blended for two minutes, then compressed into tablets using a rotary tablet press. The resulting tablets had a hardness of 3-4 kp.

A tablet was placed in a USP disintegration apparatus, in purified water at 37° C. The tablet disintegrated completely within 40 seconds.

### Example 2

### Bioavailablity Study

Orally disintegrating tablets containing 25 mg carbidopa and 250 mg levodopa, prepared according to Example 1, were tested in a bioavailability study in 36 healthy adult volunteers, administered under fasting conditions. Tablets were placed on the tongue and allowed to disintegrate for 30 seconds (no chewing and no swallowing allowed), after an overnight fast. After 30 seconds, 240 ml of room temperature water was consumed. Blood samples were taken within one hour prior to dosing (0 hour) and after dose administration at: 0.25, 0.5, 0.75, 1, 1.5, 2, 2.5, 3, 4, 5, 6, 8, 10 and 12 hours. Plasma levodopa and carbidopa concentrations were determined using validated bioanalytical methods.

Pharmacokinetic Parameters of Carbidopa/Levodopa
Orally Disintegrating Tablets, 25/250 mg

| | Tmax (hr) | Cmax (ng/ml) | AUCinf_obs (hr * ng/ml) |
|---|---|---|---|
| Carbidopa | 2.8 ± 1.0 | 98.5 ± 38.3 | 559.6 ± 197.0 |
| (Mean ± SD) | | | |
| Levodopa | 1.7 ± 0.8 | 1469.1 ± 567.2 | 4477.8 ± 1576.8 |
| (Mean ± SD) | | | |

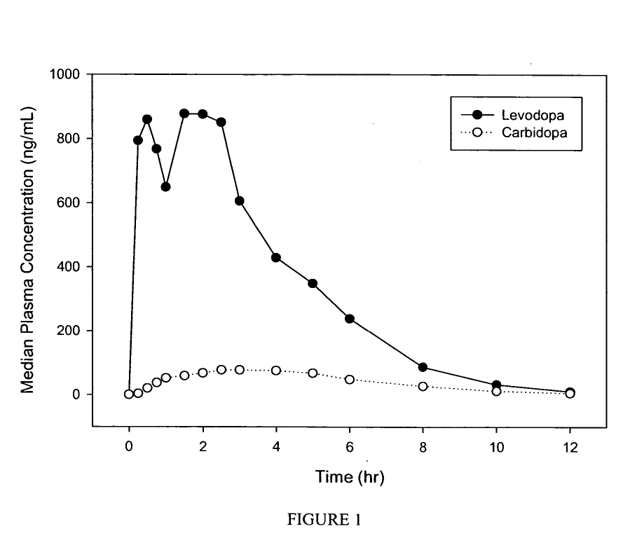

FIG. 1 shows the plasma levels of carbidopa and levodopa obtained from 25/250 mg orally disintegrating tablets according to this bioavailability study.

1. An orally disintegrating tablet prepared by a wet granulation process comprising:
(a) granulating a mixture of levodopa, carbidopa, a binder and a solvent;
(b) drying the granules from step (a);
(c) milling oversized granules;
(d) blending the granules with a lubricant, and
(e) compressing the product from step (e) into said tablet.

2. The orally disintegrating tablet according to claim 1, wherein the ratio of carbidopa to levodopa is from about 1:1 to about 1:3.

3. The orally disintegrating tablet according to claim 2, wherein the ratio of carbidopa to levodopa is about 1:1.

4. The orally disintegrating tablet according to claim 2, wherein the ratio of carbidopa to levodopa is about 1:2.

5. The orally disintegrating tablet according to claim 2, wherein the ratio of carbidopa to levodopa is about 1:3.

6. The orally disintegrating tablet according to claim 1 wherein the mixture of step (a) further includes a disintegrant.

7. The orally disintegrating tablet according to claim 1 wherein a disintegrant is blended with the granules in step (d).

8. The orally disintegrating tablet according to claim 1 wherein the mixture of step (a) further includes a diluent.

9. The orally disintegrating tablet according to claim 1, wherein the binder is selected from the group consisting of starch, alginic acid, sodium alginate, carboxymethylcellulose sodium, guar gum, hydroxylpropyl cellulose, methylcellulose, magnesium aluminum silicate, povidone, gelatin and a mixture of any thereof.

10. The orally disintegrating tablet according to claim 2, wherein the carbidopa is present in an amount of at least 25 mg.

11. The orally disintegrating tablet according to claim 6, comprising an intragranular disintegrant and an intergranular disintegrant.

12. The orally disintegrating tablet according to claim 6, wherein the disintegrant is selected from the group consisting of croscarmellose sodium, kaolin, powdered sugar, crospovidone, carboxymethylcellulose, alginic acid, sodium alginate, polacrilin potassium, sodium starch glycolate, hydroxypropyl cellulose, methylcellulose, magnesium aluminum silicate, microcrystalline cellulose and a mixture of any thereof.

13. The orally disintegrating tablet according to claim 8, wherein the diluent is selected from the group consisting of cellulose, methylcellulose, carboxymethylcellulose, microcrystalline cellulose and a mixture of any thereof.

14. The orally disintegrating tablet according to claim 9, wherein the binder comprises starch.

15. The orally disintegrating tablet according to claim 14, wherein the starch is present in an amount of about 10% to about 70% by weight of the total tablet weight

16. The orally disintegrating tablet according to claim 12, wherein the disintegrant is croscarmellose sodium.

17. The orally disintegrating tablet according to claim 13, wherein the diluent is microcrystalline cellulose.

18. The orally disintegrating tablet according to claim 17 wherein the lubricant is magnesium stearate.

19. The orally disintegrating tablet according to claim 1 wherein the tablet disintegrates within about 45 seconds in vitro in a USP disintegration apparatus in purified water.

20. An orally disintegrating tablet comprising carbidopa and levodopa in a ratio of from about 1:1 to 1:10, wherein said orally disintegrating tablet disintegrates within about 60 seconds in vitro in a USP disintegration apparatus in purified water, said orally disintegrating tablet comprising a disintegrant and a binder selected from the group consisting of starch, starch, alginic acid, sodium alginate, carboxymethylcellulose sodium, guar gum, hydroxypropyl cellulose, methylcellulose, magnesium aluminum silicate, povidone, gelatin and a mixture of any thereof.

21. The orally disintegrating tablet according to claim 20, wherein the ratio of carbidopa to levodopa is from about 1:1 to about 1:3.

22. The orally disintegrating tablet according to claim 21 wherein the amount of carbidopa is at least about 25 milligrams.

23. The orally disintegrating tablet according to claim 20, wherein said orally disintegrating tablet disintegrates within about 45 seconds in vitro in a USP disintegration apparatus in purified water.

24. The orally disintegrating tablet according to claim 20 wherein the binder comprises starch, and is present in an amount of from about 10 percent to about 70 percent by weight of the total tablet weight.

25. The orally disintegrating tablet according to claim 20, wherein the disintegrant is an intragranular disintegrant selected from the group consisting of sodium starch glycolate, croscarmellose sodium, kaolin, powdered sugar, carboxymethylcellulose, microcrystalline cellulose, sodium alginate, hydroxypropyl cellulose, methylcellulose, magnesium aluminum silicate, crospovidone and a mixture of any thereof.

26. The orally disintegrating tablet according to claim 20, further comprising microcrystalline cellulose as a diluent.

27. The orally disintegrating tablet according to claim 25 further comprising an intergranular disintegrant.

28. The orally disintegrating tablet according to claim 25, wherein the intragranular disintegrant is croscarmellose sodium.

29. A method of treating a patient having Parkinson's Disease or related disorder comprising administering an orally disintegrating tablet of claim 1.

30. The method according to claim 29, wherein the ratio of carbidopa to levodopa is from about 1:1 to about 1:3.

31. The method according to claim 30 wherein the amount of carbidopa is at least about 25 milligrams.

32. The method according to claim 30, wherein the ratio of carbidopa to levodopa is about 1:1.

33. A method of preparing an orally disintegrating tablet by wet granulation comprising the steps of:
(a) granulating a mixture of levodopa, carbidopa, a binder and a solvent;
(b) drying the granules from step (a);
(c) milling oversized granules;
(d) blending the granules with a lubricant, and
(e) compressing the product from step (e) into said tablet.

34. The method according to claim 33, wherein the ratio of carbidopa to levodopa is from about 1:1 to about 1:3.

35. The method according to claim 33 wherein the mixture of step (a) further includes a disintegrant.

36. The method according to claim 33 wherein a disintegrant is blended with the granules in step (d).

37. The method according to claim 33 wherein the mixture of step (a) further includes a diluent.

38. The method according to claim 33, wherein the binder is selected from the group consisting of starch, alginic acid, sodium alginate, carboxymethylcellulose sodium, guar gum, hydroxypropyl cellulose, methylcellulose, magnesium aluminum silicate, povidone, gelatin and a mixture of any thereof.

39. The method according to claim 34, wherein the carbidopa is present in an amount of at least 25 mg.

40. The method according to claim 35, wherein the disintegrant is selected from the group consisting of croscarmellose sodium, kaolin, powdered sugar, crospovidone, carboxymethylcellulose, alginic acid, sodium alginate, polacrilin potassium, sodium starch glycolate, hydroxypropyl cellulose, methylcellulose, magnesium aluminum silicate, microcrystalline cellulose and a mixture of any thereof.

41. The method according to claim 37, wherein the diluent is selected from the group consisting of cellulose, methylcellulose, carboxymethylcellulose, microcrystalline cellulose and a mixture of thereof.

42. The method according to claim 38, wherein the binder comprises starch.
